(19) United States
(12) Patent Application Publication
(10) Pub. No.: US 2014/0328929 A1
(43) Pub. Date: Nov. 6, 2014

(54) Method of Preventing Acute Graft-Versus-Host Disease using Oral Beclomethasone Dipropionate

(71) Applicant: Soligenix, Inc., Princeton, NJ (US)

(72) Inventors: George B. McDonald, Seattle, WA (US); Paul Martin, Seattle, WA (US); Christopher Schaber, Princeton, NJ (US)

(21) Appl. No.: 14/361,103

(22) PCT Filed: Nov. 30, 2012
(86) PCT No.: PCT/US12/67462

§ 371 Date: May 28, 2014

Related U.S. Application Data
(60) Provisional application No. 61/565,446, filed on Nov. 30, 2011.

(51) Int. Cl. A61K 31/573; A61K 45/06

(52) U.S. Cl. CPC A61K 31/573; A61K 45/06
USPC 424/489; 514/180; 514/171

(57) ABSTRACT

Results from two randomized trials have shown that oral beclomethasone dipropionate (BDP) is effective for treatment of acute gastrointestinal graft-versus-host disease (GVHD). Here, we report results of a double-blind, randomized placebo-controlled phase II study designed to test the hypothesis that acute GVHD could be prevented by administration of oral BDP, beginning before hematopoietic cell transplantation (HCT) and continuing until day 75 after HCT. Study drug (BDP or placebo) was administered as 1 mg immediate-release formulation plus 1 mg delayed-release formulation orally four times daily. According to the primary endpoint, systemic glucocorticoid treatment for GVHD was given to 60 of the 92 participants (65%) in the BDP arm, versus 31 of 46 participants (67%) in the placebo arm. The secondary efficacy endpoints showed no statistically significant differences between the two arms. The proportion of participants who took at least 90% of the prescribed study drug during the first 4 weeks after HCT was 54% overall. Lower severity of mucositis strongly correlated with higher adherence to the schedule of study drug administration. Inconsistent adherence related to mucositis during recovery after myeloablative conditioning may have obscured a beneficial therapeutic effect in the current study.

### RELATED APPLICATIONS

This application is a United States National Stage Application claiming priority under 35 U.S.C. 371 from International Patent Application No. PCT/US12/67462 filed on Nov. 30, 2012, which claims priority from U.S. Provisional Application No. 61/565,446, filed on Nov. 30, 2011, the contents of which are hereby incorporated by reference herein.

### GOVERNMENT SUPPORT

This research was supported by grant CA18029 from the National Cancer Institute and by a grant from Soligenix, Inc. S.A.P is supported by grant K23HL077446 and by an ASBMT/Viropharma New Investigator Award.

### TECHNICAL FIELD OF THE INVENTION

This invention relates to the prevention of Graft-Versus-Host Disease (GVHD), particularly the specific timing of administration of an orally effective therapeutic agent prior to transplantation and continuing for a period after transplantation.

### BACKGROUND OF THE INVENTION

GVHD is a major complication of allogeneic hematopoietic cell transplant (HCT). GVHD is an inflammatory disease initiated by T cells in the donor graft that recognize histocompatibility and other tissue antigens of the host and is mediated by a variety of effector cells and inflammatory cytokines. GVHD presents in both acute and chronic forms. The most common target organs are the skin, liver, and GI tract, and may involve other organs such as the lung. The symptoms of acute GVHD usually present within 100 days of transplantation. The symptoms of cGVHD usually present somewhat later, and up to three years after allogeneic HCT and are often proceeded by a history of acute GVHD.

In the classical sense, acute GVHD is characterized by selective damage to the liver, skin (rash), and mucosa, and the gastrointestinal tract. Newer research indicates that other graft-versus-host-disease target organs include the immune system (the hematopoietic system, e.g., the bone marrow and the thymus) itself, and the lungs in the form of idiopathic pneumonitis. Biomarkers can be used to identify specific causes of GVHD, such as elafin in the skin. Chronic GVHD also attacks the above organs, but over its long-term course can also cause damage to the connective tissue and exocrine glands.

Acute GVHD of the GI tract can result in severe intestinal inflammation, sloughing of the mucosal membrane, severe diarrhea, abdominal pain, nausea, and vomiting. This is typically diagnosed via intestinal biopsy. Liver GVHD is measured by the bilirubin level in acute patients. Skin GVHD results in a diffuse maculopapular rash, sometimes in a lacy pattern.

Acute GVHD is staged as follows: overall grade (skin-liver-gut) with each organ staged individually from a low of 1 to a high of 4. Patients with grade IV GVHD usually have a poor prognosis. If the GVHD is severe and requires intense immunosuppression involving steroids and additional agents to get under control, the patient may develop severe infections as a result of the immunosuppression and may die of infection.

Highly potent, topically active glucocorticoids such as beclomethasone dipropionate (BDP) have long been used for treatment of inflammatory diseases of the intestinal tract, respiratory tract and skin [1-3]. After oral administration for treatment of intestinal inflammation, BDP is rapidly hydrolyzed in both luminal fluid and in mucosal cells. BDP is highly susceptible to first-pass metabolism in the liver [4,5], is protein-bound, and has a rapid disposition, thereby limiting systemic effects. For treatment of intestinal mucosal inflammatory diseases, the retention of a topical glucocorticoid, such as BDP, and its active metabolites in the mucosa, with its reduced systemic bioavailability, offer major therapeutic advantages over systemic glucocorticoids such as prednisone and methylprednisolone, which have well-recognized systemic adverse effects [6,7].

Recent experience at our center has shown that 60% to 70% of patients require systemic steroid therapy for acute graft-versus-host disease (acute GVHD) after allogeneic hematopoietic cell transplantation (HCT) with a myeloablative conditioning regimen. Results of two previous randomized clinical trials indicated that the combination of oral BDP plus a 10 day course of prednisone (1 mg/kg/day) followed by a rapid 7 day taper was more effective than prednisone alone for initial treatment of grade II gastrointestinal GVHD [8,9]. These studies are also the only prospective, randomized clinical trials to suggest a survival advantage for a treatment of gastrointestinal GVHD.

Although prior studies evaluating prophylactic systemic glucocorticoids after allogeneic bone marrow transplantation have yielded inconsistent results, the data from the two randomized BDP GVHD treatment studies suggested that prophylactic oral BDP may be effective in preventing acute GVHD. Here, we report results of a double-blind, randomized placebo-controlled phase II study designed to test the hypothesis that prophylactic administration of oral BDP, beginning before transplantation and continuing until day 75 after transplantation, would both prevent acute GVHD and also reduce its severity when it occurred.

### SUMMARY OF THE INVENTION

In brief, this invention discloses a method of treatment for preventing the occurrence of acute GVHD in patients having undergone HCT.

HCT is the generic term that encompasses bone marrow transplantation, peripheral blood stem cell transplantation, umbilical vein blood transplantation, or any other source of pluripotent hematopoietic stem cells. The method includes the oral administration of an effective amount of a topically active corticosteroid (abbreviated herein as “TAC”) to a patient scheduled to undergo HCT. A representative TAC of this invention is beclomethasone dipropionate (BDP). Administration starts at the start of the transplantation conditioning regimen and continues through day 75 after HCT, thereby preventing acute GVHD, and also when GVHD does occur reducing the severity of occurrence.

As mentioned above, this invention is directed to a method of treatment for preventing acute GVHD which commonly follows HCT, as well as host-versus-graft disease (HVGD) or allograft rejection which commonly follows organ transplantation.

The method of the present invention employs oral administration of an effective amount of a topically active corticosteroid (TAC) to a patient scheduled to undergo hematopoietic cell or organ allograft transplantation.

Representative TACs include, but are not limited to, beclomethasone dipropionate, alclometasone dipropionate, budesonide, 22S budesonide, 22R budesonide, beclomethasone-17-monopropionate, clobetasol propionate, diflorasone diacetate, flunisolide, flurandrenolide, fluticasone propionate, halobetasol propionate, halcinonide, mometasone furoate, and triamcinalone acetonide. Such TACs are well known to those skilled in the field of, for example, intestinal disorders, and are commercially available from any number of sources. Suitable TACs of this invention have rapid first-pass metabolism in the intestine and liver, low systemic bioavailability, high topical activity, and rapid excretion (see, e.g., Thiesen et al., Alimentary Pharmacology & Therapeutics 10:487-496, 1996) (incorporated herein by reference).

These and other aspects of this invention will be evident upon reference to the following detailed description.

### BRIEF DESCRIPTION OF THE FIGURES

FIG. 1. Randomization and subsequent outcomes.

FIG. 2. Cumulative incidence of grades IIa-IV GVHD (top), grades IIb-IV GVHD (middle), and grades II-IV GVHD (bottom). Grade IIa GVHD is defined as gastrointestinal involvement with less than 1000 mL stool volume per day, less than 50% of body surface affected by rash, and serum total bilirubin concentration <2.0 mg/dL. Grade IIb GVHD is defined as gastrointestinal involvement with less than 1000 mL stool volume per day, with more than 50% of body surface affected by rash or total serum bilirubin concentration between 2.0 and 2.9 mg/d L.

FIG. 3. Cumulative incidence of grades IIa-IV GVHD (top), grades IIb-IV GVHD (middle), and grades III-IV GVHD (bottom) in patients categorized by adherence.

### DETAILED DESCRIPTION OF THE INVENTION

### Definitions

As used above and throughout the instant specification and appending claims, the following terms, unless otherwise indicated, shall be understood to have the following meanings:

As used herein, the term “treatment” means administration of an effective therapy to prevent symptoms associated with acute GVHD with administration beginning at the start of the transplantation conditioning regimen and continuing through day 75 following HCT. The term “traditional treatment” means administration of an effective therapy following HCT.

As used herein, the term “tissue” means intestinal mucosa or the small bile ducts in the liver. Intestinal mucosa includes mucosa of the esophagus, stomach, small intestine and colon.

As used herein, the term “patient” means a human or other mammal.

As used herein, the term “effective amount” is meant to describe an amount of a compound effective in producing the desired therapeutic effect.

### Preferred Embodiments

In one embodiment, an effective amount of a TAC is orally administered to a patient scheduled to undergo HCT and continuing through at least day 75 following HCT.

In more specific embodiments, the TAC is administered orally at a dosage of 2 mg/day to 8 mg/day in a form suitable for oral administration, such as capsules, pills, coated microspheres with specific dissolution qualities, or emulsions. Other agents may optionally also be included in such oral formulations.

In one embodiment of this invention, the TAC is beclomethasone dipropionate (BDP). BDP is a compound which is available from a number of commercial sources, such as Schering-Plough Corporation (Kenilworth, N.J.) in bulk crystalline form, and has the following structure (i.e., beclomethasone 17,21-dipropionate):

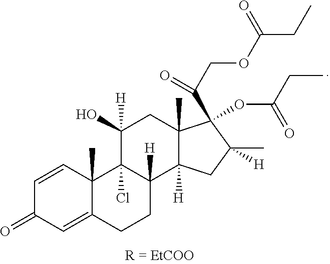

In another embodiment, the TAC is administered to patients scheduled to undergo hematopoietic cell or organ allograft transplantation, i.e. allogeneic hematopoietic cell recipients who will receive marrow-ablative chemotherapy and/or total body irradiation followed by donor hematopoietic cell infusion, or patients scheduled to undergo intestinal or liver transplantation. Such procedures have been widely disclosed, and are well known to those skilled in this field. Transplant patients receive a therapeutically acceptable amount of a TAC by oral administration. The TAC may be formulated for oral administration by techniques well known in the formulation field, including formulation as a capsule, pill, coated microsphere with specific dissolution qualities, or emulsion. Suitable capsules or pills generally contain from 1 mg to 2 mg TAC, and typically about 1 mg TAC, plus optional fillers, such as lactose, and may be coated with a variety of materials, such as cellulose acetate phthalate. By appropriate coating, such capsules, microspheres or pills may be made to dissolve within various location of the intestinal tract. For example, enteric-coated capsules prepared with a coating of cellulose acetate phthalate are known to dissolve in the alkaline environment of the small bowel, thus delivering its content to the small bowl and colon. Emulsions containing a TAC may also be employed for oral delivery, including optional emulsifying agents.

In addition to the TAC, acceptable carriers and/or diluents may be employed and are familiar to those skilled in the art. Formulations in the form of pills, capsules, microspheres, granules or tablets may contain, in addition to one or more TACs, diluents, dispersing and surface active agents, binders and lubricants. One skilled in the art may further formulate the TAC in an appropriate manner, and in accordance with accepted practices, such as those disclosed in Remington's Pharmaceutical Sciences, Gennaro, Ed., Mack Publishing Co., Easton, Pa., 1990 (incorporated herein by reference).

In the practice of this invention, a “therapeutically effective amount” of a TAC is administered to a patient in need thereof. In general terms, a therapeutically effective amount of a TAC is an amount which, when delivered orally, beginning at the start of transplantation conditioning and continuing to day 75 following hematopoietic cell transplantation. Such an amount may be readily determined by one skilled in the art by well-known dose-response investigations, and will generally range from 2 mg/day to 8 mg/day, and more typically range from 6 mg/day to 8 mg/day.

In the context of GVHD, prophylactic therapeutic administration of a TAC beings at the start of the transplantation conditioning regimen, and continues through 75 days after infusion of hematopoietic cells. An important aspect of this invention is that the TAC is orally administered such that it is topically administered to the intestinal and/or liver tissue. Thus, oral administration, as that term is used herein, is not intended to encompass systemic administration, such as by intravenous injection. Rather, the TAC has little (if any) systemic availability, but high topical activity on intestinal and or liver tissue. Such limited distribution results in fewer side effects, which is a significant advantage of this invention.

In addition to differences with regard to location and timing of administration, there is also a biological basis between the prevention and traditional treatment of symptoms of acute GVHD. Corticosteroids have been used in the management of GVHD symptoms. Conversely, prophylactic therapy using corticosteroids to prevent the occurrence of GVHD has not previously been disclosed. Therefore, using BDP as preventative therapy, the present invention is able to prevent the symptoms of acute GVHD or in the alternative greatly reduce the severity of symptoms when there is an occurrence of acute GVHD following HCT.

In traditional treatment, the objectives are to suppress a wide variety of biological events that have already resulted in tissue destruction, for example, the generation of inflammatory cytokines, the recruitment of additional inflammatory cells to the site of injury, the destruction of the barrier function of the intestinal mucosa (the lining), the passage of bacteria and toxins through the damaged intestinal mucosa, the up-regulation of biologic responses to bacteria and endotoxin, and the widespread organ responses to these events (such as leaky blood vessels, increased cardiac output, decreased systemic vascular resistance, diffuse lung injury, and renal insufficiency). When a patient has GVHD, treatment is successful only 50-75% of the time; the remainder of the patients generally dies. The present invention proposes to prevent the occurrence or severity of occurrence of acute GVHD and thereby reduce the likelihood of a patient suffering from the described biological events.

By appropriate formulation of the TAC (such as enterically coated capsules), it can be delivered to the entire mucosal surface of the intestine in high doses. Thus, TAC can achieve high concentrations in the intestinal mucosa where this alloimmune recognition event will take place. It is believed that blunting the initiating event prevents the large cascade of biologic events that make up the syndromes of acute GVHD and HVGD.

It will be appreciated that, although specific embodiments of this invention have been described herein for purpose of illustration, various modifications may be made without departing from the spirit and scope of the invention.

### Evidence of Oral BDP Prevention of Acute GVHD

### Patients.

Patients who had allogeneic hematopoietic cell transplantation with marrow or growth factor-mobilized blood cells from HLA-A, -B, -C, -DRB1, and HLA-DQB1-allele matched or single-allele or antigen mismatched related or unrelated donors after a myeloablative conditioning regimen (>800 cGy total body irradiation [TBI] and cyclophosphamide or high-dose busulfan and cyclophosphamide) were eligible for this study. Patients who had transplantation with cord blood cells and those who were enrolled in studies that involved depletion of T cells from the graft or administration of T cell-depleting antibodies before transplantation were excluded. Patients with body weight <35 kg were also excluded because lower-dose formulations of the study drug were not available. All participants gave informed consent as documents with the use of forms approved by institutional review committees at the Fred Hutchinson Cancer Research Center and Hackensack University Medical Center.

### Treatment.

All participants received a standard regimen of methotrexate and tacrolimus after transplantation as prophylaxis against acute GVHD.[10] Methotrexate was given at 15 mg/m² on day 1 after HCT and 10 mg/m² on days 3, 6 and 11. Blinded study drug (BDP or placebo) was manufactured by Soligenix, Inc. (Princeton, N.J.) and administered under IND 77,025 (PJM, sponsor-investigator) as 1 mg immediate-release formulation plus 1 mg delayed release formulation orally four times daily. Administration of the study drug began at the start of the conditioning regimen and continued through day 75 after transplantation. Patients who were not able to ingest the study drug because of mucositis continued their participation in the study. Administration of the study drug continued during systemic immunosuppressive treatment for aGVHD, but administration of the study drug was discontinued if open-label oral BDP or budesonide was administered to treat GVHD. Institutional standards were used for antibacterial and antifungal prophylaxis.

### Endpoints.

The primary pre specified endpoint of the study was development of acute GVHD with severity sufficient to require systemic immunosuppressive treatment on or before day 90 after transplantation such as: a) prednisone or its equivalent at a 1 mg/kg/day, b) systemic immunosuppressive agents other than those in the GVHD prophylactic regimen in lieu of high-dose glucocorticoids, or c) substitution of one calcineurin inhibitor for the other in the GVHD prophylactic regimen in order to treat overt GVHD. The following actions were not considered as an indication of acute GVHD with severity sufficient to require systemic immunosuppressive treatment: a) administration of high-dose glucocorticoids for indications other than acute GVHD, b) substitution of one calcineurin inhibitor for another in the GVHD prophylactic regimen in order to manage toxicity, c) increase in the dose of the calcineurin inhibitor originally prescribed for GVHD prophylaxis in order to suppress overt manifestations of acute GVHD. For purposes of the primary endpoint, extracorporeal photopheresis was considered as systemic immunosuppressive treatment, but administration of psoralen and UV irradiation was not.

Secondary pre specified endpoints included a) cumulative glucocorticoid dose (measured as prednisone equivalents) per kg body weight during the first 75 days after transplantation, b) peak and average skin, liver and gut morbidity stages and overall grades [11] to day 90 after transplantation, c) average acute GVHD index score [12] to day 90 after transplantation, d) cumulative incidence of systemic immunosuppressive treatment for acute GVHD at any time after transplantation, including extracorporeal photopheresis, e) cumulative incidence of topical therapy for acute GVHD, including psoralen and UV irradiation, hydrocortisone cream, topical tacrolimus, oral BDP, or oral dexamethasone rinses, f) cumulative incidence of biopsy-proven gastrointestinal GVHD, g) proportion of patients with grade IIa GVHD, defined as stage I gastrointestinal involvement with stage 0-2 skin involvement and no liver involvement, h) proportions of patients with grades IIb-IV GVHD, defined as stage 3 skin involvement or stage 1 liver involvement with or without stage 1 gastrointestinal involvement, i) cumulative incidence of chronic GVHD (cGVHD) requiring systemic immunosuppressive treatment, j) number of days in the hospital during the first 90 days after transplantation, k) non-relapse mortality, l) overall survival, m) survival at 200 days after transplantation, and n) survival without recurrent malignancy. In the assessment of the acute GVHD index score [12], parenteral nutrition was employed as a surrogate for oral caloric intake less than 40% of requirements.

### Statistics.

For purposes of sample size estimation, we assumed that 60% of the participants in the placebo arm would require systemic glucocorticoid treatment for acute GVHD. The trial was designed to provide 80% power and alpha=0.05 to observe a 25% difference (delta) in the incidence of acute GVHD requiring systemic immunosuppressive treatment between the oral BDP arm and the placebo arm: this effect size was based on the outcomes of the previous prospective treatment of grade IIa acute gastrointestinal GVHD studies with oral BDP. We surmised that grade IIa GVHD might be largely eliminated by prophylactic administration of BDP. Based on historical results from our center, we estimated a frequency of grade IIa GVHD at approximately 25%. A 2:1 randomization between oral BDP and placebo was selected in order to allow more robust estimation of the effect size for a variety of potential endpoints that could be considered for subsequent trials. With these nominal design parameters, we planned to evaluate 92 participants in the oral BDP arm and 46 participants in the placebo arm. An interim analysis was conducted after 69 patients were enrolled, with a plan to end the trial if the probability of rejecting the null hypothesis had been less than 33%. This criterion was not met, and the remaining patients were accrued as originally planned.

The primary endpoint was analyzed as a binomial outcome with differences evaluated by the unadjusted x² test with stratification according to donor type (related vs. unrelated) and pretransplant risk category (chronic myeloid leukemia in chronic phase, refractory anemia without excess blasts or other hematologic malignancy in remission vs. more advanced malignancy). Other endpoints were evaluated by x² test, Student's t-test, Wilcoxon rank sum test or log-rank test as appropriate, with no adjustment to the computed significance level to account for the interim analysis. Results were analyzed with data available as of Sep. 15, 2010.

### Results

### Enrollment and Patient Characteristics.

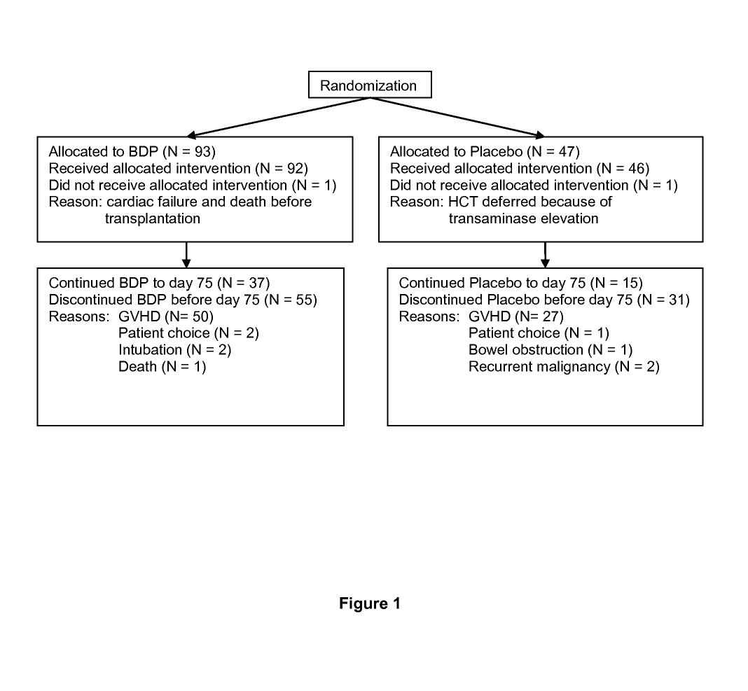

Between Aug. 14, 2007 and Apr. 14, 2010, 140 participants ranging from 8 to 63 years of age were randomized, 93 to the BDP arm and 47 to the placebo arm (FIG. 1). One patient in the BDP arm developed cardiac failure and died before the transplant, and one patient in the placebo arm was withdrawn from the study when the transplant was deferred because of severe serum alanine aminotransferase elevation after administration of cyclophosphamide. Outcomes in the remaining 92 participants in the BDP arm and 46 patients in the placebo arm are summarized below. Ten participants were enrolled at Hackensack University Medical Center, and 128 were enrolled at the Fred Hutchinson Cancer Research Center.

With one exception, characteristics of participants in the two arms of the study were well balanced (Table 1). In the BDP arm, 46 of the 92 participants (50%) received a pre-transplant conditioning regimen that included total body irradiation, compared to 15 of 46 participants (33%) in the placebo arm.

TABLE 1
Characteristics of patients, according to study arm

| | BDP | Placebo |
|---|---|---|
| Characteristic | N = 92 | N = 46 |
| Patient age, median years (range) | 44 (8-63) | 47 (12-62) |
| Diagnosis at transplant, N (%) | | |
| Acute myeloid leukemia | 42 (46) | 15 (33) |
| Acute lymphoblastic leukemia | 23 (25) | 8 (17) |
| Chronic myeloid leukemia | 6 (7) | 7 (15) |
| Myelodysplastic neoplasms or | 19 (21) | 15 (33) |
| myeloproliferative syndromes | | |
| Other | 2 (2) | 1 (2) |
| Pretransplant risk category, N (%) | | |
| Low or intermediate | 78 (85) | 40 (87) |
| High | 14 (15) | 6 (13) |
| Donor/patient gender, N (%) | | |
| Female/male | 24 (26) | 14 (30) |
| Other | 68 (74) | 32 (70) |
| High intensity conditioning | | |
| regimens, N (%) | | |
| Cyclophosphamide and total body | 46 (50) | 15 (33) |
| irradiation | | |
| Busulfan and cyclophosphamide | 46 (50) | 31 (67) |
| HLA-matching and donor type, N (%) | | |
| HLA-identical related | 33 (36) | 15 (33) |
| HLA-mismatched related | 2 (2) | 0 (0) |
| HLA-matched unrelated | 43 (47) | 25 (54) |
| HLA-mismatched unrelated | 14 (15) | 6 (13) |
| Type of graft, N (%) | | |
| Bone marrow | 23 (25) | 12 (26) |
| G-CSF-mobilized blood cells | 69 (75) | 34 (74) |
| Mean percent study drug taken through | 80 | 82 |
| week 4 after transplant | | |
| Median percent study drug taken | 92 | 93 |
| through week 4 after transplant | | |
| ≧80% adherence through week 4 | 57 (62) | 33 (72) |
| after transplant, N (%) | | |
| ≧90% adherence through week 4 | 49 (53) | 26 (57) |
| after transplant, N (%) | | |

Adherence with study drug administration during the first 4 weeks after transplantation was similar in the two arms but was less consistent than anticipated The mean adherence during the week before HCT was 94%, declined to 88% and 65% during the first and second weeks after HCT, respectively, and then recovered to 79% and 91% during the third and fourth weeks, respectively. The proportion of participants who took at least 80% of the prescribed study drug during the first 4 weeks after HCT appeared to be somewhat lower in the BDP arm (62%) than in the placebo arm (72%), but this difference was not statistically significant (P=0.26). The proportion of participants who took at least 90% of the prescribed study drug during the first 4 weeks after HCT was 53% in the BDP arm and 57% in the placebo arm.

In the BDP arm, 37 of the 92 participants (40%) took the study drug until day 75 after transplantation as planned, compared to 15 of the 46 participants (33%) in the placebo arm (P 0.38). As shown in FIG. 1, reasons for discontinuation of study drug administration before day 75 included systemic treatment for acute GVHD, patient choice, intubation, bowel obstruction, recurrent malignancy and death, with no statistically significant differences between the two study arms in the proportion of participants with premature discontinuation or in the distribution of causes.

### Assessment of Efficacy.

Systemic glucocorticoid treatment for GVHD was given to 60 participants (65%) in the BDP arm, compared to 31 of 46 participants (67%) in the placebo arm (P=0.80) (Table 2). The secondary efficacy endpoints also showed no statistically significant differences between the two arms. These included cumulative prednisone-equivalent glucocorticoid dose from the day of transplantation to day 75 after transplantation, peak and average weekly morbidity scores in the skin, liver and gastrointestinal tract, overall morbidity score, peak GVHD grade, and average weekly GVHD activity index.

TABLE 2
Efficacy endpoints, according to study arm

| | BDP | Placebo | |
|---|---|---|---|
| Endpoint | N = 92 | N = 46 | P |
| Systemic treatment for acute GVHD before day 90, N (%) | 60 (65) | 31 (67) | 0.80 |
| Cumulative mg/kg prednisone-equivalent systemic | 33.1 (23.3) | 37.0 (27.1) | 0.47 |
| glucocorticoid dose to day 75, mean (SD)* | | | |
| Peak skin morbidity score (SD) | 1.13 (1.13) | 1.26 (1.10) | 0.52 |
| Average weekly skin morbidity score (SD) | 0.30 (0.42) | 0.27 (0.36) | 0.68 |
| Peak liver morbidity score (SD) | 0.42 (0.93) | 0.26 (0.65) | 0.29 |
| Average weekly liver morbidity score (SD) | 0.11 (0.31) | 0.04 (0.12) | 0.15 |
| Peak weekly gut morbidity score (SD) | 1.93 (1.07) | 1.93 (1.06) | 0.99 |
| Average weekly gut morbidity score (SD) | 0.63 (0.36) | 0.71 (0.33) | 0.25 |
| Peak weekly gut morbidity score after day 21 (SD) | 1.39 (1.07) | 1.41 (0.97) | 0.91 |
| Average weekly gut morbidity score after day 21 (SD) | 0.47 (0.48) | 0.59 (0.50) | 0.16 |
| Peak overall morbidity score, mean (SD) | 2.54 (0.62) | 2.54 (0.59) | 0.99 |
| Average weekly overall morbidity score, mean (SD) | 1.22 (0.53) | 1.36 (0.46) | 0.14 |
| Peak GVHD grade, N (%) | | | 0.50 |
| 0-1 | 31 (34) | 15 (33) | |
| IIa | 41 (45) | 17 (37) | |
| IIb-IV | 20 (22) | 14 (30) | |
| Average weekly GVHD activity index, mean (SD) | 22.0 (13.1) | 21.7 (11.2) | 0.89 |

*for GVHD, not including steroid treatment for other indications

Efficacy outcomes assessed as hazard ratios showed no statistically significant differences between the two arms (Table 3). The hazard ratio of systemic treatment for acute GVHD in the BDP arm compared to the placebo arm was 0.92 (95% C.I. 0.6-1.4, P=0.69). Likewise, no statistically significant differences were observed in the hazards of gastrointestinal GVHD proven by biopsy, topical treatment for cutaneous or gastrointestinal GVHD, or secondary systemic treatment for GVHD. Results were similar when the analysis was adjusted for the imbalanced use of TBI between the 2 arms (data not shown).

TABLE 3
Efficacy and safety outcomes assessed as hazard ratios,
according to study arm

| | Events | | | | |
|---|---|---|---|---|---|
| Outcome | BDP | Placebo | HR | 95% CI | P |
| Efficacy | | | | | |
| Systemic treatment for | 61 | 31 | 0.92 | (0.6-1.4) | 0.69 |
| acute GVHD* | | | | | |
| Gastrointestinal GVHD | 44 | 18 | 1.26 | (0.7-2.2) | 0.40 |
| proven by biopsy* | | | | | |
| Topical treatment for | 58 | 31 | 0.89 | (0.6-1.4) | 0.60 |
| acute GVHD* | | | | | |
| Secondary systemic | 8 | 6 | 0.65 | (0.2-1.9) | 0.43 |
| treatment for GVHD* | | | | | |
| Safety | | | | | |
| CMV viremia or disease* | 23 | 12 | 0.93 | (0.5-1.9) | 0.84 |
| Invasive yeast or mold | 1 | 1 | 0.50 | (0.0-7.9) | 0.62 |
| infection* | | | | | |
| Chronic GVHD requiring | 31 | 13 | 1.05 | (0.6-2.0) | 0.87 |
| systemic treatment* | | | | | |
| Death without recurrent | 10 | 3 | 1.60 | (0.4-5.8) | 0.46 |
| malignancy | | | | | |
| Recurrent malignancy | 19 | 12 | 0.77 | (0.4-1.6) | 0.48 |
| Death from any cause | 22 | 11 | 0.95 | (0.5-2.0) | 0.89 |
| Death or recurrent malignancy | 29 | 15 | 0.93 | (0.5-1.7) | 0.83 |

*withdrawal from follow-up or recurrent malignancy treated as competing event

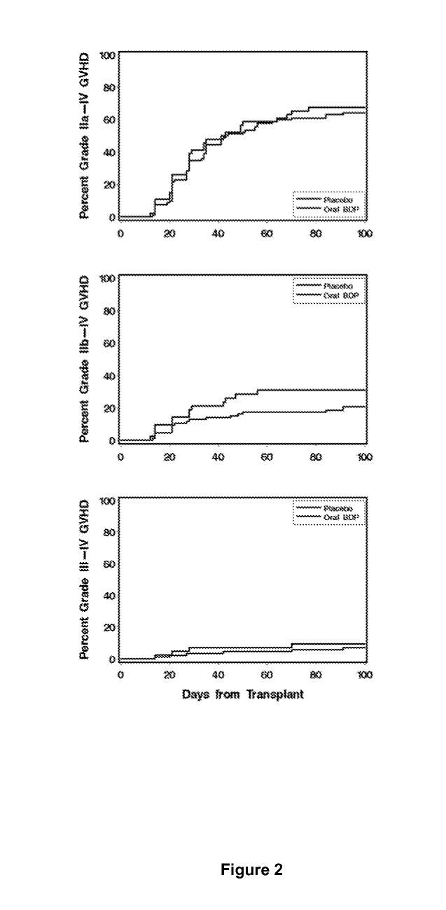

The cumulative incidence of grades IIa-IV GVHD at day 100 after transplantation was 66% (95% CI, 57%-76%) in the BDP arm and 67% (95% CI, 54%-81%) in the placebo arm (FIG. 2). The cumulative incidence of grades IIb-IV GVHD at day 100 after transplantation was 22% (95% CI, 13%-30%) in the BDP arm and 30% (95% CI, 17%-44%) in the placebo arm. The cumulative incidence of grades III-IV GVHD was 7% (95% CI, 1%-12%) in the BDP arm and 9% (95% CI, 1%-17%) in the placebo arm.

### Assessment of Safety.

A variety of adverse outcomes assessed as hazard ratios showed no statistically significant differences between the two arms (Table 3). These included the hazards of cytomegalovirus and invasive yeast or mold infection, cGVHD, death without recurrent malignancy, recurrent malignancy, death from any cause, and the composite of death or recurrent malignancy. Likewise, the incidence of gastrointestinal adverse events showed no statistically significant differences between the two arms (Table 4). Gastrointestinal infection was diagnosed in 22 participants (24%) in the BDP arm, compared to 14 participants (30%) in the placebo arm (P=0.41). Other gastrointestinal adverse events occurred infrequently.

TABLE 4
Gastrointestinal adverse events, according to study arm*

| | BDP | Placebo | |
|---|---|---|---|
| Adverse Event, N (%) | N = 92 | N = 46 | P |
| Oral infection | 3 (3) | 3 (7) | 0.37 |
| GI infection (any) | 22 (24) | 14 (30) | 0.41 |
| CMV enteritis | 2 (2) | 3 (7) | 0.20 |
| C. difficile | 16 (17) | 10 (22) | 0.54 |
| Other viral infection | 2 (2) | 1 (2) | 0.99 |
| Yeast or mold | 0 (0) | 0 (0) | |
| Other | 3 (3) | 0 (0) | 0.22 |
| Esophagitis | 8 (9) | 3 (7) | 0.67 |
| Gastritis | 2 (2) | 0 (0) | 0.32 |
| Enteritis | 0 (0) | 0 (0) | |
| Typhlitis | 1 (1) | 0 (0) | 0.48 |
| Colitis | 0 (0) | 1 (2) | 0.15 |
| GI hemorrhage | 2 (2) | 1 (2) | 0.99 |
| GI ulceration or erosion | 5 (5) | 0 (0) | 0.11 |
| Ileus | 1 (0) | 0 (0) | 0.48 |

*numbers of patients with at least one episode; excludes 2 patients who withdrew before transplantation

Gastrointestinal ulceration or erosion was diagnosed by endoscopy in 5 participants who developed anorexia, nausea or vomiting, all in the BDP arm. Four of the 5 patients with upper gastrointestinal ulceration or erosion were taking oral BDP up to the day of endoscopy, but one patient had not taken BDP for at least one month before the endoscopy. One patient was admitted to the hospital, in part due to difficulty swallowing. Gastrointestinal ulceration or erosion was not associated with other outcomes that would categorize these events as serious. Ulceration or erosion was observed in the esophagus (N=3), antrum (N=1) or both (N=1). In 3 of the endoscopy reports, ulceration or erosion was attributed to mucositis or esophagitis, and in the other 2 reports, no attribution was given. Pathology review showed GVHD in 2 cases, nonspecific inflammation in 2 cases, and no diagnostic abnormality in 1 case.

GVHD, bacteremia and recurrent malignancy were the most frequent serious adverse events. Other serious adverse events occurred at frequencies <5%. The two arms were somewhat imbalanced in the number of serious adverse events related to bacteremia, with 14 in the BDP arm and 5 in the placebo arm. For this reason, we evaluated the distribution of all bacteremia episodes. Because participants took the study drug until day 75 after transplantation and then stopped, we included all bacteremia episodes that occurred before day 103. We observed 12 episodes of Enterococcus bacteremia in 10 of the 92 participants in the BDP arm: 9 before day 17 after transplantation and the remaining 3 after day 57. We observed a single episode of Enterococcus bacteremia among the 46 participants in the placebo arm, on day 15 after transplantation. In contrast, we observed 15 episodes of gram-negative bacteremia in the BDP arm, compared to 7 in the placebo arm, which approximates the 2:1 distribution of participants between the two arms.

Time-to-first event analyses were used to evaluate the risks of bacteremia in the BDP arm compared to the placebo arm. Follow-up was censored at 103 days after HCT, 28 days after the end of study drug administration. The hazard ratios were 1.38 (95% CI 0.8-2.5) for any bacteremia, 1.32 (95% CI 0.7-2.5) for gram-positive bacteremia, and 5.24 (95% CI 0.7-41) for Enterococcus bacteremia. Enterococcus bacteremia episodes occurred in 10 (9 BDP, 1 placebo) of the 61 participants who received total body irradiation before transplantation and in 1 (BDP) of the 77 participants who received busulfan and cyclophosphamide before transplantation (P<0.001, Fisher's exact test).

The two arms were also somewhat imbalanced in the number of serious adverse events related to mucositis, with 5 in the BDP arm and 1 in the placebo arm. For this reason, we evaluated the severity of mucositis in the two arms for the 118 patients who had evaluations by an oral medicine specialist on at least one occasion after transplantation at the FHCRC. This analysis did not include participants who enrolled in the study at Hackensack University because these patients did not have evaluations by an oral medicine specialist. The mean maximum recorded oral mucositis index (OMI) scores [13] were 22.1±14.2 (SD) for the 79 participants in the BDP arm and 22.7±12.6 for the 39 participants in the placebo arm (P=0.82, t-test).

Adrenal Function at Day 75 after Transplantation.

Cosyntropin stimulation tests were administered at approximately 75 days after transplantation in order to determine whether the level of systemic glucocorticoid activity associated with prolonged oral administration of BDP was sufficient to decrease adrenal function in patients who did not receive systemic glucocorticoid treatment. Abnormal adrenal function was detected in 26 of the 30 participants (87%) tested in the BDP arm, compared to 5 of the 8 participants (63%) tested in the placebo arm (P=0.12). Basal cortisol plasma concentrations and the mean fold-increase in cortisol concentrations at 30 minutes after cosyntropin stimulation showed no statistically significant differences between the two arms.

### Exploratory Analyses.

The lack of apparent efficacy of oral BDP in preventing acute GVHD was unexpected, and adherence to the study therapy was lower than anticipated. A number of exploratory analyses were done in an attempt to clarify these findings and to investigate whether the two might be associated. Adherence to the schedule of study drug administration might have been compromised by mucositis, which is commonly observed after myeloablative conditioning. Lower severity of oral mucositis as measured by the peak OMI score was correlated with increased adherence (Spearman's correlation coefficient −0.38, P<0.0001). Adherence was higher in patients who did not receive systemic treatment for GVHD compared to those who received systemic treatment in the BDP arm (P=0.001, t-test) but not in the placebo arm (P=0.98), consistent with the possibility that reduced adherence may have compromised the BDP treatment effect. Among the 50 BDP patients with ≧90% adherence, 27 (54%) had systemic treatment for GVHD, versus 31 (67%) of the 46 placebo patients with ≧90% adherence (Table 5).

TABLE 5
Primary Efficacy endpoints

| | | Placebo | |
|---|---|---|---|
| Endpoint | | N = 46 | P |
| | BDP | | |
| | N = 92 | | |
| Systemic treatment for acute GVHD | 62 (67) | 32 (70) | 0.76 |
| before day 90, N (%) | | | |
| PRIMARY ENDPOINT | | | |
| | BDP | | |
| | N = 50 | | |
| BDP >90% adherent group requiring systemic | 27 (54) | 31(67) | 0.18 |
| treatment for acute GVHD before day 90, | | | |
| N (%) | | | |

The mean cumulative dose of prednisone through day 75 post HCT was 1145 mg among all BOP patients with ≧90% adherence, versus 1807 mg among all placebo patients with ≧90% adherence. The mean of the average dose of prednisone through day 75 post HCT was 15.4 mg among all BDP patients with ≧90% adherence, versus 23.7 mg among all placebo patients with ≧90% adherence, reflecting the greater ability to reduce prednisone if the patient adheres to the BDP regimen (Table 6).

TABLE 6
Prednisone Dosing Outcomes

| Cumulative Prednisone | | Cumulative |
|---|---|---|
| Dose through Day 75 Post HCT | Number | Dose |
| Placebo | 46 | 1807 mg |
| BDP <50% adherent | 8 | 2048 mg |
| BDP 50-90% adherent | 34 | 2105 mg |
| BDP >90% adherent | 50 | 1145 mg |
| Mean of the Average | | Average |
| Dose through Day 75 Post HCT | Number | Mean |
| Placebo | 46 | 23.7 mg |
| BDP <50% adherent | 8 | 29.2 mg |
| BDP 50-90% adherent | 34 | 28.7 mg |
| BDP >90% adherent | 50 | 15.4 mg |

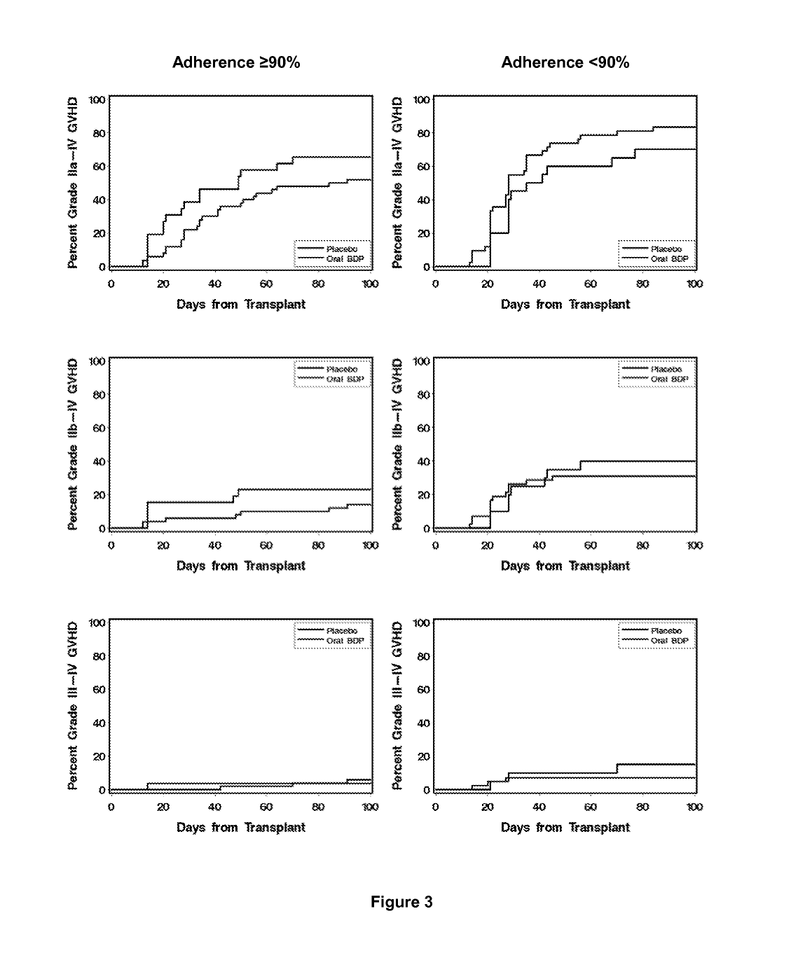

As shown in FIG. 3, the cumulative incidence of grades IIa-IV GVHD (top), grades IIb-IV GVHD (middle), and grades II-IV GVHD (bottom) in patients categorized according to adherence (≧90% versus <90%) is consistent with the possibility that BDP had a beneficial effect in preventing grade IIa-IV GVHD and grade IIb-IV GVHD in those with better adherence.

To investigate the potential relationship between mucositis and response to BDP, we assessed outcomes in subsets of participants categorized according to the peak OMI score above and below the median. Thirty-nine of the 59 participants (66%) with peak OMI scores ≦20 had ≧90% adherence with study drug administration, compared to only 23 of the 59 participants (39%) with peak OMI scores >20. Thirty-seven of the 59 participants (63%) with peak OMI scores ≦20 had grades IIa-III GVHD, compared to 45 of the 59 participants (76%) with peak OMI scores >20. Nine of the 43 participants (21%) with peak OMI scores ≦20 in the BDP arm had grades IIb-III GVHD, compared to 6 of the 16 participants (38%) with peak OMI scores ≦20 in the placebo arm. Seven of the 36 participants (19%) with peak OMI scores >20 in the BDP arm had grades IIb-III GVHD, compared to 6 of the 23 participants (26%) with peak OMI scores >20 in the placebo arm.

The overall mortality data also suggest an improvement over placebo, which was 14% among all BDP patients with ≧90% adherence, versus 24% among all placebo patients with ≧90% adherence (Table 7).

TABLE 7
Overall Mortality

| | BDP | Placebo | |
|---|---|---|---|
| Endpoint | N = 50 | N = 46 | P |
| BDP >90% adherent group | 7 (14) | 11 (24) | 0.19 |
| Overall Mortality, N (%) | | | |

Similarly, the incidence of relapse was reduced among all BDP patients with ≧90% adherence (18%) versus the incidence of relapse among all placebo patients with ≧90% adherence (26%), further suggesting the importance of adherence to the BDP regimen through the treatment protocol (Table 8).

TABLE 8
Relapse

| | BDP | Placebo | |
|---|---|---|---|
| Endpoint | N = 50 | N = 46 | P |
| BDP >90% adherent group Relapse, N (%) | 9 (18) | 12 (26) | 0.30 |

Results of this study did not support the hypothesis that prophylactic oral administration of BDP decreases the risk of acute GVHD as assessed either by the primary endpoint or by any of the pre-specified secondary endpoints. Observations in the two previous randomized trials where adherence was consistently >90% indicated that oral BDP is effective for treatment of acute gastrointestinal GVHD [8,9]. Inconsistent study drug adherence related to mucositis during recovery after myeloablative conditioning may have obscured a beneficial therapeutic effect in the current study. Poor adherence is unlikely to have affected outcomes among participants in the placebo arm but might have negated a positive result among those in the BDP arm, particularly with respect to the incidence of grades IIa-IV and grades IIb-IV GVHD.

The 5 previous randomized trials evaluating the effects of prophylactic systemic glucocorticoid administration after allogeneic bone marrow transplantation have varied results. The risk of grades II-IV GVHD was decreased in 2 studies, unaffected in 2, and increased in 1 [14-18]. The study that showed an increased risk of grades II-IV GVHD differed from the others in that administration of methylprednisolone started on the day of transplantation [14]. In the other studies, steroid administration began on day 7 or 14. It was suggested that steroid administration during the first 11 days after transplantation could have interfered with the GVHD-preventive effects of methotrexate administered on days 1, 3, 6 and 11 after HCT. Additional results in the same study suggested that steroid administration beginning on day 15 after transplantation did not increase the risk of GVHD, but also did not produce any benefit [14].

Although our surveillance of adverse effects in this study confirmed results of previous studies in showing that oral BDP is well tolerated during the first 2 to 3 months after allogeneic hematopoietic cell transplantation, attention to gastric ulceration or erosions and Enterococcus bacteremia is warranted in future studies. The number of patients and the frequencies of these events in the current study were not sufficient to demonstrate that these adverse events were caused by BDP. Previous studies did not show an increased risk of bacteremia associated with the use of systemic glucocorticoids for prevention of GVHD [14, 16-18]. The association of BDP administration with Enterococcus bacteremia in the current study is not statistically significant, is clearly data-driven and could be spurious. We were unable to identify any previous reports of such an association. All but one of the Enterococcus bacteremia episodes occurred in patients who had received TBI, and most of the episodes occurred during the 2^{nd} and 3^{rd} weeks after transplantation. Future studies should examine the possibility that administration of BDP interferes with protective commensal bacteria or innate defense mechanisms against Enterococcus species in the intestinal tract during recovery after total body irradiation.

The double-blind design is a major strength of our study, but the interpretation of results is hampered by limited statistical power and by the inability of participants to maintain high levels of adherence with oral ingestion because of mucositis during the first several weeks after myeloablative conditioning. The study was designed to evaluate only 138 patients, based on the anticipation that the effect size would be ˜25%. Our results do not rule out the possibility that prophylactic administration of BDP might provide benefits of smaller magnitude, or that alternative administration schedules r testing in patients treated with nonmyeloablative conditioning regimens before HCT could demonstrate benefits that were not observed in the present study.

### REFERENCES

1. Levine, D S. Immune modulating therapies for idiopathic inflammatory bowel disease. Adv Pharmacol. 1994; 25:171-234.
2. Rutgeerts P, Lofberg R, Malchow H, et al. A comparison of budesonide with prednisolone for active Crohn's disease. N Engl J Med. 1994; 331:842-845.
3. Lofberg R, Ostergaard-Thomsen O, Langholz E, et al. Budesonide versus prednisolone retention enemas in active distal ulcerative colitis. Aliment Pharmacol Therapeut. 1994; 8:623-629.
4. Martin L E, Tanner R J N, Clark T J H, Cochrane G M. Absorption and metabolism of orally administered beclomethasone dipropionate. Clin Pharmacol Ther. 1974; 15:267-275.
5. Oishi T, Deguchi T, Marumo H. Metabolic fate of beclomethasone 17,21-dipropionate in the rat. Pharmacomet. 1981; 22:717-727.
6. Boeckh M, Nichols W G, Papanicolaou G, Rubin R, Wingard J R, Zaia J. Cytomegalovirus in hematopoietic stem cell transplant recipients: Current status, known challenges, and future strategies. Biol Blood Marrow Transplant. 2003; 9:543-558.
7. Garcia-Vidal C, Upton A, Kirby K A, Marr K A. Epidemiology of invasive mold infections in allogeneic stem cell transplant recipients: biological risk factors for infection according to time after transplantation. Clinical Infectious Diseases. 2008; 47:1041-1050.
8. McDonald G B, Bouvier M, Hockenbery D M, et al. Oral beclomethasone dipropionate for treatment of intestinal graft-versus-host disease: a randomized, controlled trial. Gastroenterology. 1998 115:28-35.
9. Hockenbery D M, Cruickshank S, Rodell T C, et al. A randomized, placebo-controlled trial of oral beclomethasone dipropionate as a prednisone-sparing therapy for gastrointestinal graft-versus-host disease. Blood. 2007; 109:4557-4563.
10. Ratanatharathorn V, Nash R A, Przepiorka D, et al. Phase III study comparing methotrexate and tacrolimus (Prograf®, FK506) with methotrexate and cyclosporine for graft-versus-host prophylaxis after HLA-identical sibling bone marrow transplantation. Blood. 1998; 92:2303-2314.
11. Al-Ghamdi H, Leisenring W, Bensinger W I, et al. A proposed objective way to assess results of randomized prospective clinical trials with acute graft-versus-host disease as an outcome of interest. Brit J Haematol. 2001; 113:461-469.
12. Leisenring W M, Martin P J, Petersdorf E W, et al. An acute graft-versus-host disease activity index to predict survival after hematopoietic cell transplantation with myeloablative conditioning regimens. Blood. 2006; 108: 749-755.
13. McGuire D B, Peterson D E, Muller S, Owen D C, Slemmons M F, Schubert M M. The 20 item oral mucositis index: reliability and validity in bone marrow and stem cell transplant patients. Cancer Investigation. 2002; 20:893-903.
14. Storb R, Pepe M, Anasetti C, et al. What role for prednisone in prevention of acute graft-versus-host disease in patients undergoing marrow transplantation. Blood. 1990; 76:1037-1045.
15. Atkinson K, Biggs J, Concannon A, et al. A prospective randomized trial of cyclosporine and methotrexate versus cyclosporine, methotrexate and prednisolone for prevention of graft-versus-host disease after HLA-identical sibling marrow transplantation for haematological malignancy. Aust New Zea J Med. 1991; 21:850-856.
16. Deeg H J, Lin D, Leisenring W, et al. Cyclosporine or cyclosporine plus methotrexate for prophylaxis of graft-versus-host disease: a prospective, randomized trial. Blood. 1997; 89:3880-3887.
17. Chao, N J, Synder D S, Jain M, et al. Equivalence of 2 effective graft-versus-host disease prophylaxis regimens: results of a prospective double-blind trial. Biol Blood Marrow Transplant. 2000; 6:254-261.
18. Ruutu T, Volin L, Parkkali T, Juvonen E, Elonen E. Cyclosporine, methotrexate, and prednisolone compared with cyclosporine and methotrexate for the prevention of graft-versus-host disease in bone marrow transplantation from HLA-identical sibling donor: a prospective randomized study. Blood. 2000; 96:2391-2398.

We claim:

1. A method of preventing the occurrence of acute GVHD in a patient requiring hematopoietic cell transplantation, the method comprising prophylactic topically active oral administration of beclomethasone dipropionate (BDP) wherein treatment is directed to tissue selected from the group consisting of intestine and liver and further wherein the BDP is initially administered prior to hematopoietic cell transplantation (HCT) and continuing at least through day 75 post transplantation.

2. The method of claim 1 wherein the BDP is administered daily at a dosage of 2 mg per day to 8 mg per day.

3. The method of claim 1 wherein the BDP is formulated for oral administration in the form of a pill, capsule or microsphere.

4. The method of claim 1 wherein the BDP is formulated such that the pill, microsphere, or capsule dissolves in the stomach, small intestine or colon.

5. The method of claim 1 wherein the BDP is formulated for oral administration in the form of an emulsion.

6. The method of claim 1 wherein the patient is a candidate for receiving HLA-mismatched hematopoietic stem cells.

7. The method of claim 1 wherein the patient is a candidate for receiving unrelated donor hematopoietic stem cells, umbilical vein hematopoietic stem cells, or peripheral blood stem cells.

8. The method of claim 1 wherein the BDP is administered in combination with other prophylactic agents.

9. A method of reducing the severity of acute GVHD occurrence in a patient requiring hematopoietic cell transplantation, the method comprising prophylactic topically active oral administration of BDP wherein treatment is directed to tissue selected from the group consisting of intestine and liver and further wherein the BDP is initially administered prior to HCT and continuing at least through day 75 post transplantation.

10. The method of claim 9 wherein the BDP is administered daily at a dosage of 2 mg per day to 8 mg per day.

11. The method of claim 9 wherein the BDP is formulated for oral administration in the form of a pill, capsule or microsphere.

12. The method of claim 9 wherein the BDP is formulated such that the pill, microsphere, or capsule dissolves in the stomach, small intestine or colon.

13. The method of claim 9 wherein the BDP is formulated for oral administration in the form of an emulsion.

14. The method of claim 9 wherein the patient is a candidate for receiving HLA-mismatched hematopoietic stem cells.

15. The method of claim 9 wherein the patient is a candidate for receiving unrelated donor hematopoietic stem cells, umbilical vein hematopoietic stem cells, or peripheral blood stem cells.

16. The method of claim 9 wherein the BDP is administered in combination with other prophylactic agents
